(19) United States
(12) Patent Application Publication
(10) Pub. No.: US 2016/0045337 A1
(43) Pub. Date: Feb. 18, 2016

(54) BASE PLATE AND BLADE DESIGN FOR A LEG PROSTHETIC

(71) Applicant: Altair Engineering, Inc., Troy, MI (US)

(72) Inventors: CRAIG MACKIEWICZ, CLAWSON, MI (US); MICHAEL JAMES PREWITT, Lincoln Park, MI (US); KEVIN ROBERT SHINN, EDENVILLE, MI (US); JUSTIN ROBERT SHINN, Edenville, MI (US); ERICK SHINICHI IKEDA, Culver City, CA (US); JACQUES MAGLOIRE PERRAULT, BEND, OR (US); BENJAMIN DOUGLAS HEFNER, Birmingham, MI (US); LARRY JAMES PARKER, III, Bloomfield Township, MI (US); BRADLEY JAMES HASSBERGER, Rochester, MI (US); EDWARD FREDERICK WETTLAUFER, JR., St. Clair Shores, MI (US)

(21) Appl. No.: 14/824,363

(22) Filed: Aug. 12, 2015

Related U.S. Application Data
(60) Provisional application No. 62/036,835, filed on Aug. 13, 2014.
(60) Provisional application No. 62/036,837, filed on Aug. 13, 2014.
(60) Provisional application No. 62/036,840, filed on Aug. 13, 2014.
(60) Provisional application No. 62/036,842, filed on Aug. 13, 2014.
(60) Provisional application No. 62/036,843, filed on Aug. 13, 2014.

(51) Int. Cl. A61F 2/60; A61F 2/80

(52) U.S. Cl. CPC A61F 2/60; A61F 2/80; A61F 2002/5016

(57) ABSTRACT

A blade-type prosthesis assembly includes a blade body extending between a blade bottom and a blade top. The blade top is securable to a socket for a limb and the blade bottom defines a convex surface formed by at least one edge of the blade bottom curving toward the blade top. The prosthesis assembly further includes a base plate. The base plate includes a base plate top surface securable to the blade bottom, a base plate bottom surface opposing the base plate top surface, and a plurality of ground-engaging elements extending from the base plate bottom surface.

### CROSS REFERENCE TO RELATED APPLICATIONS

This application claims priority to U.S. Provisional Application Ser. No. 62/036,835 filed Aug. 13, 2014, U.S. Provisional Application Ser. No. 62/036,837 filed Aug. 13, 2014, U.S. Provisional Application Ser. No. 62/036,840 filed Aug. 13, 2014, U.S. Provisional Application Ser. No. 62/036,842 filed Aug. 13, 2014, and U.S. Provisional Application Ser. No. 62/036,843 filed Aug. 13, 2014 which are all incorporated herein by reference in their entirety.

This application is related to co-pending application Ser. No. 14/824,386 filed Aug. 12, 2015 (Attorney Docket No. AEI-390-B) entitled “BLADE SHROUD DESIGN FOR A LEG PROSTHETIC” and to co-pending application Ser. No. 14/824,419 filed Aug. 12, 2015 (Attorney Docket No. AEI-392-B) entitled “SUIT DESIGN FOR A LEG PROSTHETIC” which are both incorporated herein by reference in their entirety.

### FIELD OF THE DISCLOSURE

The embodiments disclosed herein relate generally to prosthetics, and more specifically, to blade shape and base plates for blade-type leg prosthetics.

### BACKGROUND

A variety of prosthetics and limb enhancements have been developed for both aesthetic and functional needs, including leg prosthetics and enhancements to aid wearers in activities such as walking, performing job functions, and playing sports. One group of leg prosthetics in this category includes trans-tibial prosthetics, often referred to as below the knee (BK) leg prosthetics. These have come to include blade-type leg prosthetics, also known as “flex-foot cheetah” prosthetics, for athletic use. Although blade-type leg prosthetics are otherwise satisfactory, wearers of blade-type leg prosthetics may desire improvements in how force is transferred to a ground surface when performing activities.

### SUMMARY

In one aspect, a blade for a blade-type prosthesis assembly comprises a blade body extending between a blade bottom and a blade top. The blade top is securable to a socket for a limb and the blade bottom defines a convex surface formed by at least one edge of the blade bottom curving toward the blade top.

In another aspect, a base plate for a blade-type prosthesis assembly comprises a base plate top surface securable to a blade bottom of a blade body. The blade body extends from the blade bottom to a blade top securable to a socket for a limb. The base plate further includes a base plate bottom surface opposing the base plate top surface and a plurality of ground-engaging elements extending from the base plate bottom surface.

In another aspect, a blade-type prosthesis assembly comprises a blade body extending between a blade bottom and a blade top. The blade top is securable to a socket for a limb. The assembly further includes a base plate comprising a base plate top surface securable to the blade bottom, a base plate bottom surface opposing the base plate top surface, and a plurality of ground-engaging elements extending from the base plate bottom surface.

These and other aspects will be discussed in additional detail below.

### BRIEF DESCRIPTION OF THE DRAWINGS

The description herein makes reference to the accompanying drawings wherein like reference numerals refer to like parts throughout the several views, and wherein:

FIG. 1 is a partially exploded side view of a below the knee blade-type leg prosthesis assembly including a socket, blade, and an example of a base plate;

FIG. 2 is a perspective view of another example of a blade for a blade-type leg prosthesis assembly;

FIGS. 3A-3B are perspective and front views of the blade of FIG. 1 showing a twist near a blade top and a convex portion near a blade bottom;

FIGS. 4A-4E are bottom views of various base plate examples for a leg prosthesis assembly.

FIG. 5 is a partial side view of another example of a base plate installed on a blade; and

FIGS. 6A-6B are partially exploded perspective views of exemplary adapters for attaching exemplary base plates to the blade of FIG. 2.

### DETAILED DESCRIPTION

Blade designs and base plates for blade-type leg prosthetics are disclosed herein. The blade or the base plate may include a curved and/or contoured bottom surface to aid in lateral movement. Such blade and base plate designs may allow for improved transfer of energy from a wearer to a ground surface. The base plate may be removable and may be adapted to flex or change orientation as the blade is deformed. The base plate may also include a bottom surface design with various gripping features such as spikes, cleats, cups, nubs, grooves, or scoops in a pattern and location configured to optimize the use of the blade-type leg prosthetic depending on the wearer's activity.

As used herein, the terminology “prosthesis” or “prosthetic” may indicate any artificial limb or limb enhancement, including upper extremity enhancements, lower extremity trans-tibial and trans-femoral prostheses, or other lower extremity enhancements. The non-limiting examples disclosed herein describe blade-type leg prosthetics, but it is contemplated that the features described may be utilized with a variety of prosthetics or enhancements known to those skilled in the art.

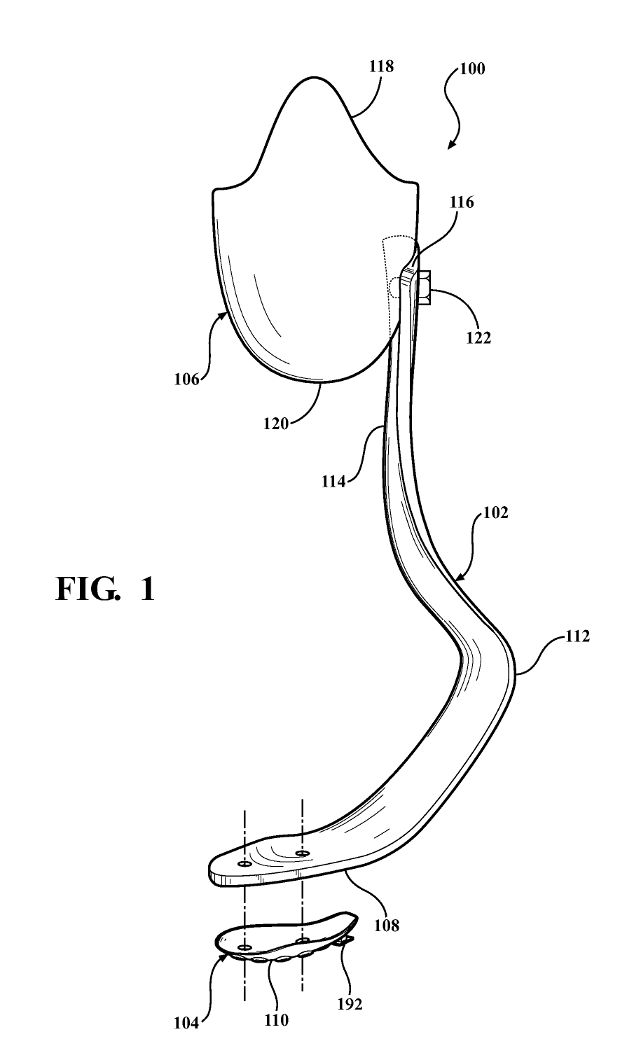

FIG. 1 is a partially exploded side view of a below the knee blade-type leg prosthesis assembly including a socket, blade, and an example of a base plate. The prosthesis assembly 100 may be described as generally comprising blade 102, base plate 104, and socket 106. Prosthesis assembly 100 is shown as a trans-tibial prosthetic, and often referred to as a below the knee (BK) prosthetic, but it is contemplated that embodiments of base plate 104 may be utilized with a variety of prosthetic devices or limb enhancements.

Blade 102 may include blade bottom 108, a first inflexion 112, a second inflexion 114, and blade top 116. Blade inflexions 112, 114 may allow blade 102 to compress when a load is applied in certain directions. For example, blade 102 may compress and expand during walking or running. Blade 102 may be constructed from a variety of natural or synthetic materials capable of withstanding forces associated with walking, running, or other wearer activities, such as metal, rubber, and polymer. For example, blade 102 may be made out of a carbon fiber reinforced polymer. Blade 102 may be used either with or without base plate 104.

Base plate 104 may be fixedly attached to blade 102 at a blade bottom 108 or may be capable of being removably attached and detached using a variety of attachment components. For example, base plate 104 may be attachable to blade 102 using an adapter (described below), sliding engagement, bolts, clips, pins, screws, adhesive, or straps. In the example shown in FIG. 1, a set of holes is present in both base plate 104 and blade bottom 108 sufficient for bolts, pins, or other means to be threaded through both base plate 104 and blade bottom 108 to connect base plate 104 to blade bottom 108. In some embodiments, base plate 104 may attach and capture a portion of blade 102 while contacting both a top and bottom surface of blade bottom 108.

Removable and interchangeable base plates 104 may allow a wearer to customize prosthesis assembly 100 depending on the wearer's activity type and/or the ground surface characteristics experienced by the wearer during the activity. Further discussed below, base plate 104 may have a bottom surface 110 including shapes and structures designed to provide a desired interaction with the ground surface for a specific activity. Further, base plate 104 can include a hook 192 providing attachment means for a garment or serving as a retaining means for storage of base plate 104.

Socket 106 may include an open socket top 118 and a closed socket bottom 120. Socket 106 may be substantially hollow, having a uniform or varying thickness. Socket top 118 may be sized accordingly to receive at least a portion of a wearer's limb. Socket 106 may be attached to a wearer during use through a variety of methods. In some embodiments, friction based attachment features may be used such as straps or clips configured to attach to a garment on a wearer. In some embodiments, suction based attachments may be utilized, such as a sock or sleeve designed to extend over socket 106 and a wearer's limb. For example, a method of attachment may include a wearer placing socket 106 at the end of a limb and attaching socket 106 by pulling a compression sock over socket 106 in a direction from socket bottom 120 to socket top 118 and onto the wearer's limb.

In some embodiments, socket 106 may be attachable to blade 102 at blade top 116 using socket attachment members 122. For example, attachment members 122 may be pins or bolts configured to extend through apertures defined in blade top 116. Socket 106 may be constructed out of any natural or synthetic material capable of substantially retaining its shape, such as metals and polymers. For example, socket 106 may be formed from carbon fiber reinforced polymer and may be formed in a custom shape to match a particular wearer's partial limb.

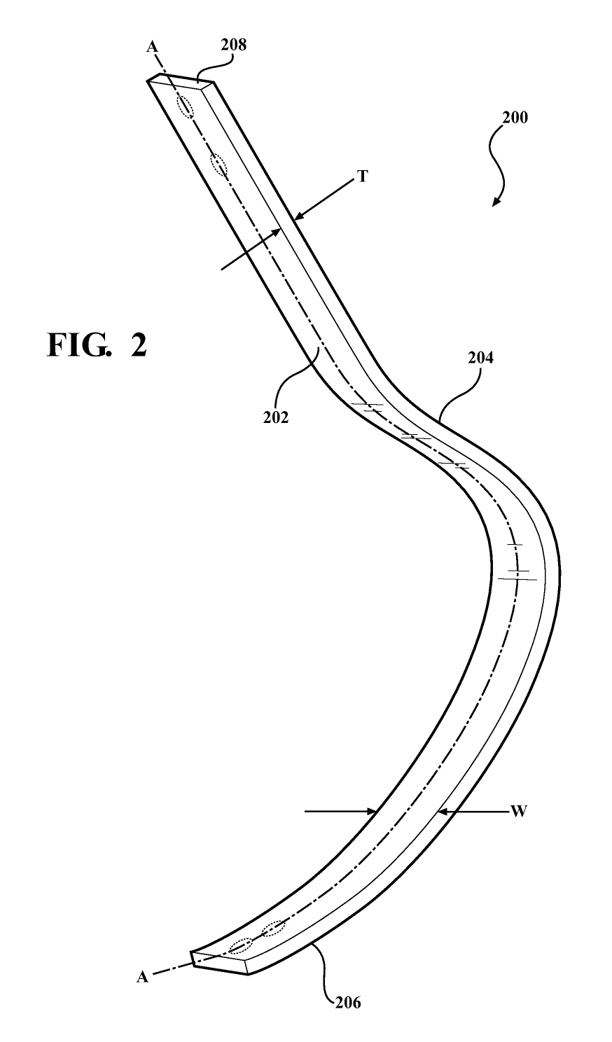

FIG. 2 is a perspective view of another example of a blade for a blade-type leg prosthesis assembly 100. Blade 200 may define a front face 202 and a rear face 204. In some embodiments, front face 202 may oppose and be substantially parallel to rear face 204, where thickness T may define a distance between front face 202 and rear face 204. It is also contemplated that front face 202 and rear face 204 may be angled with respect to each other, or that thickness T may vary along the length of blade 200. Blade 200 may have a maximum width W. In some embodiments, width W may remain constant along blade 200 as illustrated in FIG. 2.

In the example shown in FIG. 2, portions of blade 200 may not twist or rotate about its length, with front face 202 being substantially perpendicular to a plane orthogonal to longitudinal axis A. In other words, in this example, a blade bottom 206 and a blade top 208 of blade 200 remain untwisted. Furthermore, blade 200 of FIG. 2 may be designed such that axis A lies in a common plane, with blade 200 being symmetrical about the common plane including axis A, such as may be suitable for left and right side leg prosthetics.

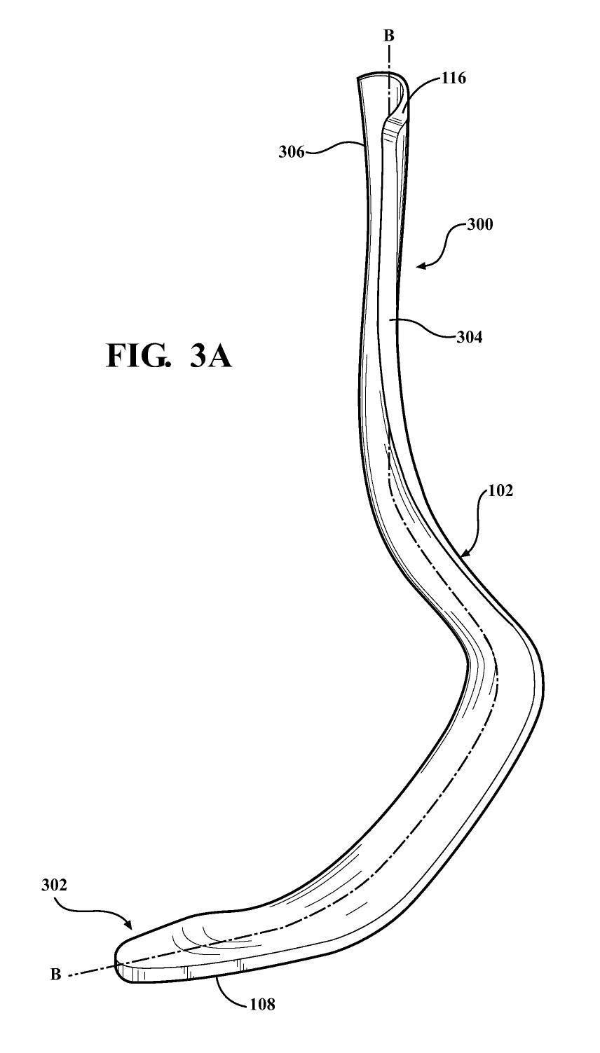

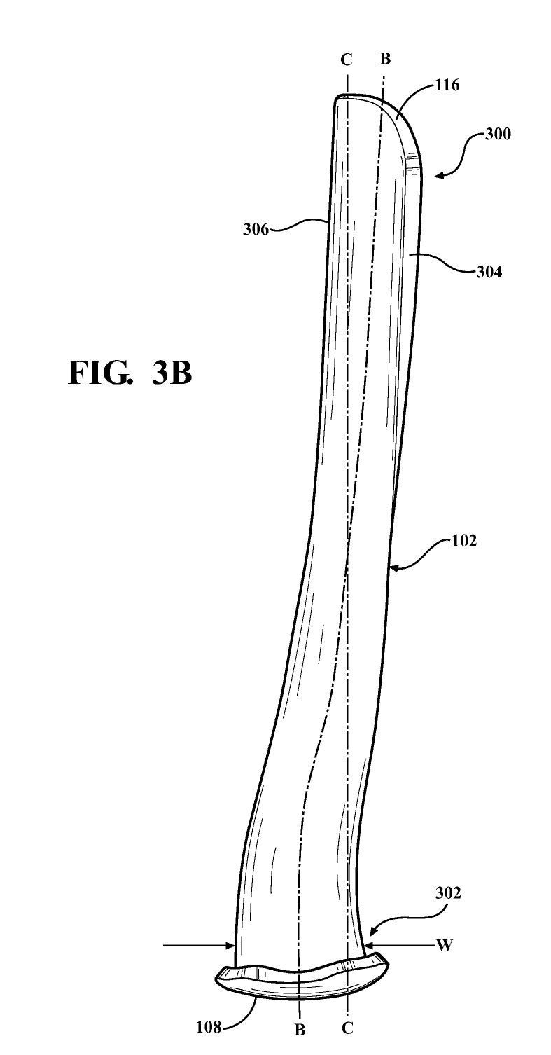

FIGS. 3A-3B are perspective and front views of blade 102 of FIG. 1 showing a twist 300 near blade top 116 and a convex portion 302 near blade bottom 108. Twists located along blade 102 may aid in alignment for a wearer while walking, running, jumping, etc., may increase strength or rigidity in certain portions, or may allow for easier connections to socket 106. Twist 300, located near blade top 116, may be present where blade 102 pivots or rotates such that an inside edge 304 extends forward and an outside edge 306 extends rearward when the blade 102 is coupled to the socket 106 of FIG. 1. Twist 300 occurs as the edges 304, 306 are rotated forward and backward about an axis B extending along a centerline of a body of the blade 102. Though FIGS. 3A & 3B show twist 300 located near blade top 116, one or more twists or curves may be located at varying positions along blade 102.

In addition to twisting, front and rear surfaces of blade 102 may bend, angle, or curve. For example, blade top 116 of blade 102 may curve such that front and rear surfaces produce concave shapes facing a forward and outer direction. This may be best illustrated by the curve in blade top 116 shown in FIG. 3A. The inclusion of curves, angles, or bends in blade 102 may provide additional strength to portions of blade 102, aid in alignment while walking, running, or playing sports, or aid in the attachment to socket 106. Having curves or twists, such as twist 300 near blade top 116 may allow the blade to connect to socket 106 through a rotational or twist connection. This may eliminate the need for other fastener types, be less burdensome on a wearer, or reduce time involved in attaching or detaching blades.

In addition to curves or twists located near blade top 116, blade 102 may include convex portion 302 at blade bottom 108. Convex portion 302 may be formed by lateral edges of blade bottom 108 curving away from a bottom most portion of blade bottom 108 toward blade top 116. Convex portion 302 may allow blade 102 to have increased contact portions with a ground surface when blade 102 is angled when positioned against the ground surface. In other words, blade bottom 108 having curved convex portion 302 may mimic the human ankle which provides rotation for proper planting of a foot into the ground regardless of the impact angle. This may allow blade bottom 108 to have enough friction and surface area to be used in prosthesis assembly 100 with or without base plate 104.

In the examples of FIGS. 3A & 3B, the width of blade 102 varies along the length of blade 102. In some embodiments, maximum width W may occur near blade bottom 108, with the width of blade 102 increasing along the length of blade 102 from blade top 116 to blade bottom 108. This may decrease material and weight of blade 102 while increasing strength and contact area near blade bottom 108. It is contemplated that the thickness of blade 102 may also vary along a length and a width of blade 102 or remain constant.

In some embodiments, blade 102 may be shaped such that axis B does not lie in a common plane. Instead, blade bottom 108 extends further in a lateral direction than blade top 116. That is, blade 102 may be shaped such that the blade bottom 108 may angle either inward or outward in relation to an opposing leg or prosthetic to, for example, improve balance during use. Midpoints of the blade bottom 108 and the blade top 116 are thus spaced from a vertical axis C extending through a midpoint of the blade body during use of the blade-type prosthesis assembly. For example, FIG. 3B shows that a midpoint of blade bottom 108, that is, a portion of blade bottom 108 at the axis B location, is outside a midpoint of blade top 116 in a lateral direction by comparing axis B to axis C. This orientation of the blade 102 may help stability and cornering for a wearer during walking, running, or other activities.

The specific optimized shape of the blade 102 for a given wearer may be determined using a computer-based method. In some embodiments, a model is created to represent prosthesis assembly 100 for a wearer. Variables such as length of blade, movements required, particular ground surfaces and a walking or running pattern of the wearer may impact the design of the blade 102. For example, a computer-based model may be created for a wearer that includes simulated walking and running. It may be determined what contours, twists, and concave and convex portions would be ideal for the particular application.

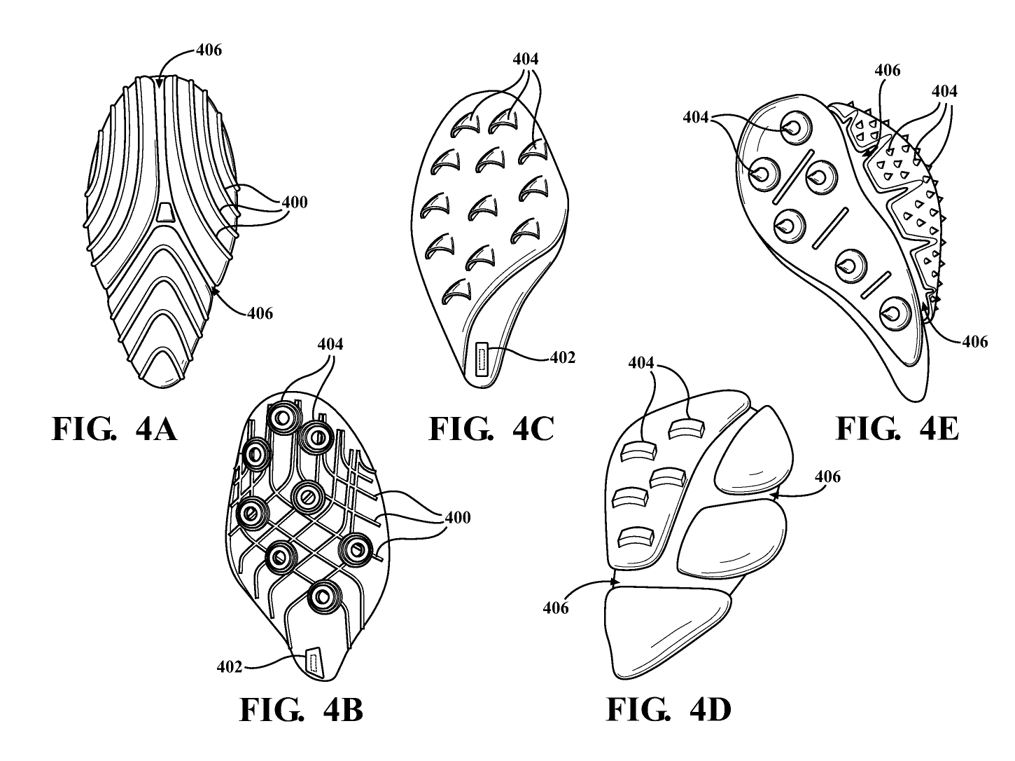

FIGS. 4A-4E are bottom views of various base plate examples for leg prosthesis assembly 100. In some examples, such as the embodiments shown in FIGS. 4D and 4E, the base plates may be asymmetrical about a plane extending through a centerline of the blade body of either blade 102 or blade 200 when the base plates are secured to either blade bottom 108 or blade bottom 206 during use of the prosthesis assembly 100 by the wearer. Asymmetry is useful when the base plates are specifically configured for use in either a right leg or left leg prosthesis assembly 100 to assist the wearer in applying force to the ground surface in way that more closely resembles a human foot and ground interaction.

It is contemplated that different shapes and material selections for the base plates may be useful to illicit different desired interactions with the ground surface. In some embodiments, the base plates may be interchangeable depending on the ground surface and wearer activity. For example, ground surfaces such as uneven terrain, tile, carpet, sand, mud, gravel, grass, turf, dirt, rocks, hard track, soft track, and pavement all have unique surface characteristics and certain base plate designs may be chosen for the particular application.

In some examples, such as the embodiments shown in FIGS. 4A-4C, the base plates may curve away from bottom surfaces toward blade tops 116, 208 near outer edges of the base plates during use of the prosthesis assembly 100 by the wearer. The curved or convex portion may allow the base plates to have a greater contact area with the ground surface when prosthesis assembly 100 is orientated at an angle to the ground surface, such as when the wearer is turning or leaning.

In some embodiments, such as the embodiment shown in FIGS. 4A and 4B, the base plates may include ribs 400 located on and extending from bottom surfaces of the base plates. Ribs 400 may provide structural support in certain directions while allowing the base plates to remain flexible. Additionally, ribs 400 may provide increased grip and friction between the bottom surfaces and the ground surface.

In some embodiments, such as the embodiments shown in FIGS. 4B and 4C, the base plates may include a hook 402, that is, a retainer element, located near a rear-most portion of a bottom surface of the base plates. Hook 402 may extend outward from the bottom surfaces and provide attachment means on the surface for use with, for example, a blade shroud attachable to prosthesis assembly 100 or a garment, such as pants or a body suit, designed to retain the prosthesis assembly 100 to the wearer. Hook 402 may also be useful in storage of the base plates, allowing the base plates to be retained against a storage surface or collected or looped together by any type of storage element.

In some embodiments, such as the embodiments shown in FIGS. 4B-4E, the base plates may include one or more ground-engaging elements 404 extending from bottom surfaces toward the ground during use of prosthesis assembly 100. Ground-engaging elements 404 may aid a wearer of prosthesis assembly 100 in gripping a ground surface to provide stability, power, traction, and cornering ability depending on the wearer's activity.

Ground-engaging elements 404 may be fixed or may be removable, such as through a threaded interaction with the bottom surfaces of the base plates or other attachment means. Ground-engaging elements 404 may be formed from a variety of materials, such as metals and polymers. Ground-engaging elements 404 may be formed in a variety of shapes and sizes. For example, ground-engaging elements 404 may be spikes or nubs as in FIG. 4E, cleats as in FIG. 4D, scoops as in FIG. 4C, cups as in FIG. 4B, or a variety of other shapes.

In some embodiments, such as the embodiments shown in FIGS. 4A, 4D, and 4E, the base plates may include one or more grooves 406 configured to allow the base plate to flex when the body of blades 102, 200 is deformed during use of the base plates with prosthesis assembly 100. Various ridges or ribs 400, ground-engaging elements 404, and grooves 406 may be positioned in certain configurations such that the alignment or location provides beneficial interaction in the form of flexing and gripping between the respective base plate and a ground surface during use of the prosthesis assembly 100. Though specific configurations of ridges 400, ground-engaging elements 404, and grooves 406 are shown in FIGS. 4A-4E, other configurations are also possible.

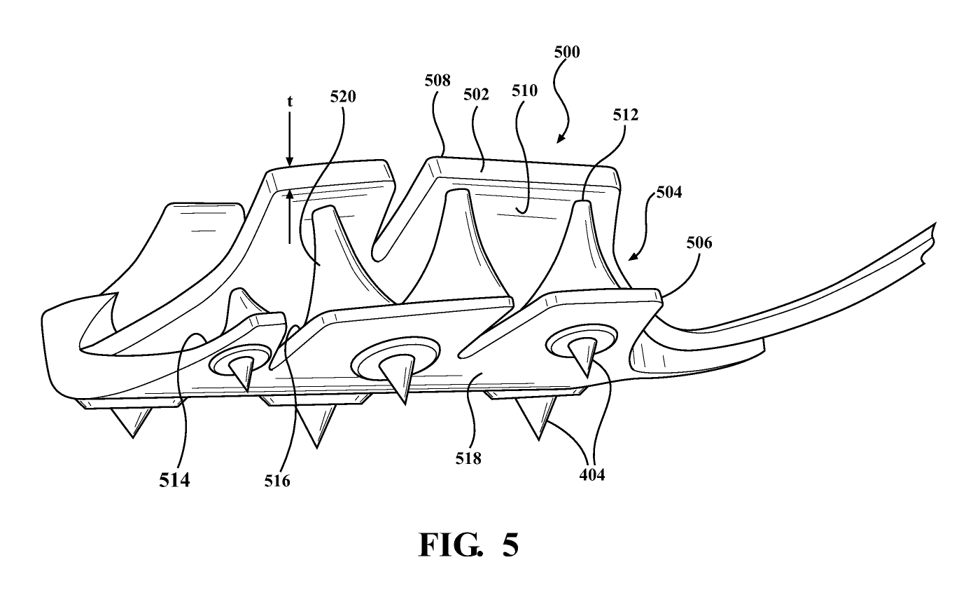

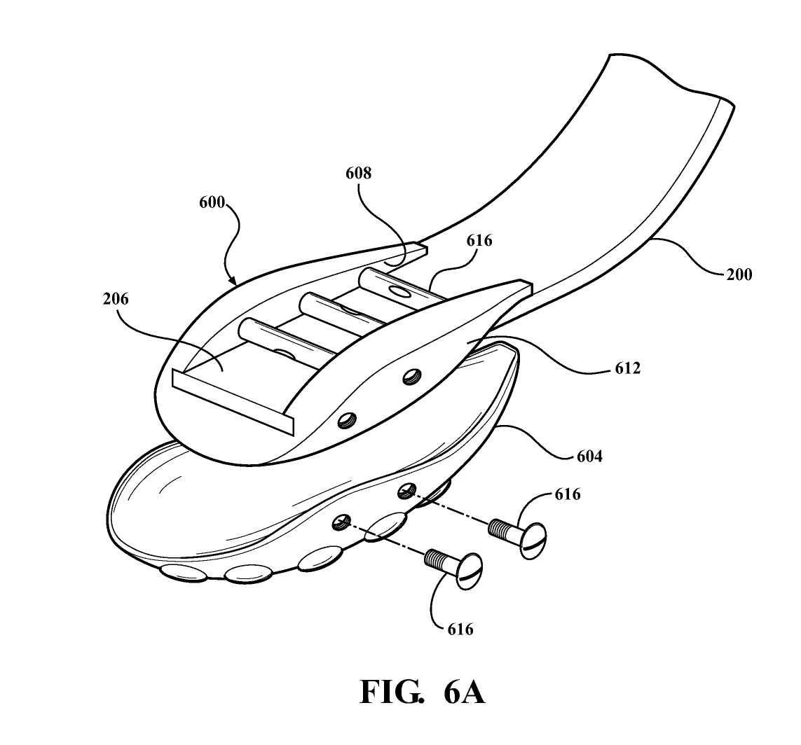

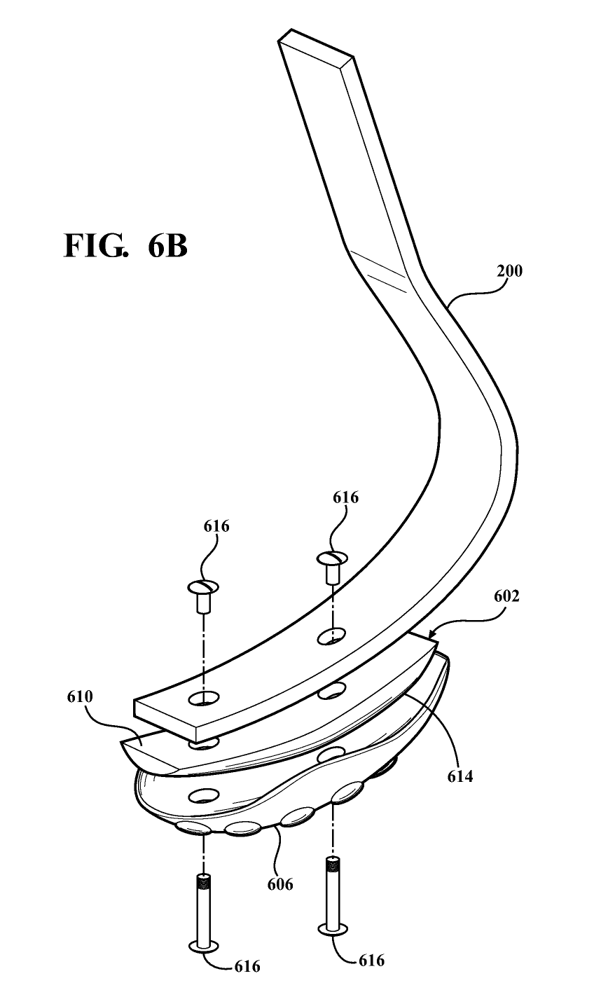

In some embodiments, top surfaces of the base plates may be shaped to interface, for example, with a substantially flat surface such as is present on blade bottom 206 of blade 200 or with a convex surface such as is present on blade bottom 108 of blade 102. The top surfaces of the base plates may also be designed such that an adapter, such as the adapters described below in reference to FIGS. 6A and 6B, is required for attachment to blade 102 or blade 200. Alternatively, an adapter may be integrally designed as part of the top surface of the base plates. FIG. 5 is a partial perspective view of another example base plate 500. Base plate 500 may be constructed of multiple layers having differing material characteristics. In some embodiments, base plate 500 may include a first layer 502, a second layer 504, and a third layer 506. First layer 502 may include first surface 508 that faces and sits in abutment with a bottom surface of either blade 102 or blade 200 when base plate 500 is attached to blade 102 or blade 200. First layer 502 may also include second surface 510 opposing and spaced a distance from first surface 508. In some embodiments, second layer 504 may be disposed between first layer 502 and third layer 506. Second layer 504 may have first surface 512 that contacts second surface 510 of first layer 502. In some embodiments, third layer 506 may have a first surface 514 in contact with a second surface 516 of second layer 504. Third layer 506 may also have second surface 518 that includes ground-engaging elements 404 of the type described in FIGS. 4A-4E.

As illustrated in FIG. 5, the thickness of base plate 500, or individual layers, may remain constant or may vary along base plate 500. For example, first layer 502 may have a constant thickness t as shown. Second layer 504 may have a thickness greater near side portions of base plate 500 that decreases along base plate 500 moving towards the center of base plate 500.

The basic structure of base plate 500 may vary depending on the particular application, and several natural and synthetic materials known by those skilled in the art may be used. In some embodiments, base plate 500 may be at least partially formed from natural rubber, polyurethane, or polyvinyl chloride (PVC) compounds. Different portions of base plate 500 may have different material characteristics. For example, first and third layers 502, 506 of base plate 500 may be composed of high durometer polymer or rubber to ensure durability, while second layer 504 may be less dense or formed of lower durometer foam to cushion or absorb energy resulting from impacts with a ground surface. The durometer of the various layers 502, 504, 506 can vary depending on the activity for which the base plate 500 is designed.

It is contemplated that the shape and structure of base plate 500 may vary depending on application. In some embodiments, second layer 504 may include gaps, channels, spaces, apertures, or other voids to reduce weight or increase compliance or cushioning. For example, second layer 504 may comprise a collection of conically shaped stems 520 extending from third layer 506 to first layer 502. In some embodiments, a bottom surface of base plate 500 may be non-planar, curved, or angled. For example, the bottom surface of base plate 500 may follow the contour of either blade 102 or blade 200. In some embodiments, the bottom surface of base plate 500 may be curved or angled in lateral directions as well.

FIGS. 6A and 6B are partially exploded side views of exemplary adapters 600, 602 for attaching exemplary base plates 604, 606 to blade 200 of FIG. 2. The base plates 604, 606 may be similar to any of the base plates of FIGS. 4A-4E. The adapters 600, 602 include adapter top surfaces 608, 610 securable to blade bottom 206 and adapter bottom surfaces 612, 614 securable to base plate top surfaces.

Adapter top surfaces 608, 610 may be generally flat surfaces such that edges of adapter top surfaces 608, 610 lie within an adapter top surface plane. The adapter top surface plane may follow the contour of front face 202 of blade 200 in the case of adapter 600 or rear face 204 of blade 200 in the case of adapter 602. Have generally flat adapter top surfaces 608, 610 allows for ease of attachment to the generally flat front face 202 or rear face 204 of blade 200.

Adapter bottom surfaces 612, 614 may be convex surfaces formed by the edges of the adapter bottom surfaces 612, 614 curving toward blade top 208 of blade 200 during use of the prosthesis assembly 100. Having generally flat adapter top surfaces 608, 610 and generally curved or convex adapter bottom surfaces 612, 614 allows adapters 600, 602 to be used to affix curved or convex base plates 604, 606 to generally flat blade 200.

Adapters 600, 602 may be affixed to blade 200 using a variety of attachment elements 616. In FIG. 6A, attachment elements 616 include bolts spanning blade bottom 206 and extending into adapter 600. In FIG. 6B, attachment elements 616 include screws and threaded caps. Other types of attachment elements 616 are also contemplated. In some examples, such as in FIG. 6B, common attachment elements 616 may be used to attach adapter 602 and base plate 606 at the same time through a set of aligned openings. In other examples, such as in FIG. 6A, attachment elements 616 differ between adapter 600 and base plate 604.

The above-described embodiments have been described in order to allow easy understanding of the invention and do not limit the invention. On the contrary, the invention is intended to cover various modifications and equivalent arrangements included within the scope of the appended claims, which scope is to be accorded the broadest interpretation so as to encompass all such modifications and equivalent structure as is permitted under the law.

What is claimed is:

1. A blade for a blade-type prosthesis assembly, comprising:
a blade body extending between a blade bottom and a blade top;
wherein the blade top is securable to a socket for a limb; and
wherein the blade bottom defines a convex surface formed by at least one edge of the blade bottom curving toward the blade top.

2. The blade of claim 1, wherein the blade body has a blade width varying between the blade bottom and the blade top.

3. The blade of claim 1, wherein the blade width is a maximum at the blade bottom and a minimum at the blade top.

4. The blade of claim 1, wherein the blade body is symmetrical about a plane extending through a centerline of the blade body.

5. The blade of claim 1, wherein midpoints of the blade bottom and the blade top are spaced from a vertical axis extending through a midpoint of the blade body during use of the blade-type prosthesis assembly.

6. The blade of claim 1, wherein the blade body includes a twisted portion rotated about an axis extending along a centerline of the blade body.

7. The blade of claim 6, wherein the twisted portion is proximate to the blade top.

8. A base plate for a blade-type prosthesis assembly, comprising:
a base plate top surface securable to a blade bottom of a blade body, the blade body extending from the blade bottom to a blade top securable to a socket for a limb;
a base plate bottom surface opposing the base plate top surface; and
a plurality of ground-engaging elements extending from the base plate bottom surface.

9. The base plate of claim 8, wherein the base plate is asymmetrical about a plane extending through a centerline of the blade body when the base plate is secured to the blade bottom.

10. The base plate of claim 8, wherein the ground-engaging elements include at least one of spikes, cleats, scoops, grooves, nubs, cups, and ridges.

11. The base plate of claim 8, wherein the base plate bottom surface defines a plurality of grooves configured to allow the base plate to flex when the blade body is deformed during use of the prosthesis assembly.

12. The base plate of claim 8, wherein the base plate further comprises:
a layer defining interstitial spaces disposed between the base plate bottom surface and the base plate top surface.

13. The base plate of claim 8, wherein the base plate further comprises a retainer element disposed on the base plate bottom surface.

14. The base plate of claim 8, wherein the base plate bottom surface defines a convex surface, the convex surface formed by at least one edge of the base plate bottom surface curving toward the blade top during use of the prosthesis assembly.

15. A blade-type prosthesis assembly, comprising:
a blade body extending between a blade bottom and a blade top, the blade top securable to a socket for a limb; and
a base plate, comprising:
a base plate top surface securable to the blade bottom;
a base plate bottom surface opposing the base plate top surface; and
a plurality of ground-engaging elements extending from the base plate bottom surface.

16. The blade-type prosthesis assembly of claim 15, wherein the base plate is removable and interchangeable with a plurality of base plates.

17. The blade-type prosthesis assembly of claim 15, wherein the ground-engaging elements include at least one of spikes, cleats, scoops, grooves, nubs, cups, and ridges.

18. The blade-type prosthesis of assembly of claim 15, wherein the base plate bottom surface defines a plurality of grooves configured to allow the base plate to flex when the blade body is deformed during use of the prosthesis assembly.

19. The blade-type prosthesis assembly of claim 15, further comprising:
an adapter, comprising:
an adapter top surface securable to the blade bottom; and
an adapter bottom surface securable to the base plate top surface;
wherein the adapter bottom surface defines a convex surface formed by at least one edge of the adapter bottom surface curving toward the blade top during use of the prosthesis assembly.

20. The blade-type prosthesis assembly of claim 19, wherein the adapter top surface defines a generally flat surface such that edges of the adapter top surface lie within an adapter top surface plane.
